(19) United States
(12) Patent Application Publication
(10) Pub. No.: US 2009/0210032 A1
(43) Pub. Date: Aug. 20, 2009

(54) MANUFACTURING METHODS, TESTING METHODS, AND TESTERS FOR INTRA-ORAL ELECTRONICALLY EMBEDDED DEVICES

(76) Inventors: Ben Zion Beiski, Kiryat-Ono (IL); Andy Wolff, Harutzim (IL)

Correspondence Address: Cozen O''Connor, 250 PARK AVENUE, NEW YORK, NY, 10177, US

(21) Appl. No.: 11/572,527

(22) PCT Filed: Jul. 20, 2005
(86) PCT No.: PCT/IL05/00773

§ 371 Date: Mar. 18, 2009

Related U.S. Application Data
(60) Provisional application No. 60/590,211, filed on Jul. 23, 2004.

(51) Int. Cl. A61N 1/08; B29C 39/02; A61N 1/36; G06F 19/00

(52) U.S. Cl. 607/59; 264/16; 607/60; 702/19

(57) ABSTRACT

The invention is directed to manufacturing and testing methods of electronic intraoral devices for diagnose, monitor and treat local and systemic diseases and conditions for humans and animals. More specifically, the current invention deals with manufacturing techniques, testing methods and a testing apparatus of mainly three types of intra-oral devices: (a) electro-stimulators for various applications such as treatment of dry mouth by stimulating saliva secretion, apnea, sleeping disorders, eating disorders (obesity, anorexia, etc.) dysphagia and others, (b) drug delivery devices; and (c) bio-sensing and monitoring devices. The common parts or the devices are: (1) art electronic module embedded in the device: (2) or a power source being embedded in the device; (3) the devices (or part of them) being placed in the oral cavity.

### BACKGROUND OF THE INVENTION

1. Field of the Invention

The present invention relates generally to:
1. Manufacturing and assembly methods of intra-oral devices for humans and animals
2. A mouthpiece for salivary glands electro-stimulator
3. Testing methods & testing techniques of intra-oral devices for humans and animals
4. Test apparatus of intra-oral devices for humans and animals

The present invention reaches the above mentioned categories for mainly three types of intra-oral devices; (a) electro-stimulators for various applications such as treatment of dry mouth by stimulating saliva secretion, apnea sleeping disorders, eating disorders (obesity, anorexia, etc.), dysphagia and others: (h) drug delivery devices: and (c) bio-sensing and monitoring devices. The common parts of categories Nos. 1 and 2 are: (a) an electronic module is embedded into the device; (b) a power source is embedded into the device; (c) the above devices for part of them) are placed in the oral cavity.

Due to the complexity of the devices, testing, programming and upgrades are often required in order to minimize the risk of placing a nonfunctional partial functional or non-customized device in order to tailor the device characteristics to the patient's needs. Those duties can be conducted at the manufacturing phase, at the clinician site and in the operating theater, in addition, placing an electronic module, including a battery (primary or secondary), inside the intra-oral environment requires unique manufacturing methods and resting methods in order to guarantee the functionality and durability of the device over time. Any object placed within the oral cavity must withstand (a) constant wetness (of saliva and intake liquids), (b) mastication forces, (c) forces applied by the tongue and other oral muscles, (h) varying pH levels from 1 to 9 usually and (e) ambient temperature of 37° C. and temperature variation ranging between +5° C. and up to +65° C. due to cold and hot drinks intake.

2. Background Information and Description of the Related Art

Testing, calibrating and programming of these electronically based devices are essential methods to guarantee electronic-based product proper functionality. Programming the device to match the patient's specific characteristics such as medical stains, age, weight, gender, DNA, origin is an option needed in few intra-oral devices. Due to the complexity of the devices, testing, programming and upgrades are often required in order to minimize the risk of placing a non-functional, partial functional or non-customized device. Those duties can be conducted at the manufacturing phase, at the clinician site and in the operating theater and in some cases also by the patient himself.

Salivary Glands Electro-Stimulators

Chronic Xerostomia (dry mouth) can be caused by Sjögren's syndrome and by other chronic diseases, nerve damage, certain medications or therapeutic irradiation. It can cause difficulty in eating dry foods, swallowing, speaking and wearing dentures; and being susceptible to dental caries, oral pain and frequent infections. Proponents of electro-stimulation as a treatment option postulate that stimulating the vicinity of the lingual nerve will result in impulses to all residual salivary tissues, major and minor, in the oral and pharyngeal regions, thus causing an increase in salivation.

In prior art, electronically based modules were placed within the oral cavity for short periods of time and were connected to extracorporeal devices. Placing a self-contained, salivary gland stimulator inside the oral cavity for long periods of time (minutes and up) and without continuous professional care requires applying unique manufacturing method and rigorous testing methods to assure the proper functionality of the device and patient's safety over time under the various dally life activities.

Salivary Glands Electro-Stimulators

Increasing secretion of saliva by electro-stimulation was described by several patents over the years. To mention few:
U.S. Pat. No. 6,230,052 “Device and method for stimulating salivation” an electro simulator supported on a dental implant. A device with built-in microprocessor, stimulating electronic modules and power source, at a size of a tooth crown which is placed on top of a dental implant. The electronics of such a device has to be ‘woken up’ from a low power consumption mode to an active mode, and the functionality of the device, such as electrical pulses patterns, battery strength and Infra Red communication, has to be tested.
PCT Application No, WO 02/000522A2 and WO 02/060522A3 by Pines et al for a removable electro-simulator device to activate the secretion of salivary glands.
U.S. Pat. Nos. 4,519,400 and 4,637,405 “Salitron” is a device manufactured by Biosonics, Inc. (PA 19034, USA), it uses an electrical probe in the mouth to stimulate the salivary glands to produce more saliva.

Other currently known applications of intra-oral devices incorporating electronically-based elements are e.g.:

Intra-Oral Electromuscular Stimulation Devices and Methods to Treat Breathing Disorders

Intra-oral electromuscular stimulation devices and methods (U.S. Pat. Nos. 6,212,433 and 6,618,627). This is an intra-oral electromuscular stimulation advice to trout breathing disorders. The stimulation device includes electrodes placed in several locations such as sublingual location posterior to a frenulum and proximate to a first molar, a second molar and a third molar of a patient. In addition, it includes a sensor that detects a respiratory parameter of a patient and outputs a signal indicative thereof. A control unit receives the signal from the sensor, distinguishes between inspiration and expiration, and initiates an electrical stimulation at a stimulation time prior to onset of inspiration and continues stimulation through a portion of inspiration at a level sufficient to induce muscle contraction without pain. The sensors, controls, electrodes, batteries have to be tested, programmed and upgraded.

Vestibular stimulation system and method (U.S. Pat. No. 6,314,324). This apparatus and method stimulates the portions of the labyrinth associated with the labyrinthine sense and/or the nerves associated therewith to perform at least one of the following functions: augment or control a patients respiratory function, open the patient's airway, induce sleep, and/or counteract vertigo, in one embodiment, the vestibular stimulating system of the invention includes 1) a stimulation element that performs the actual stimulation of the tissue, 2) a sensor to detect a physiological condition of the patient, and 3) a power/control unit that receives the signals provided by the sensor and causes stimulation energy to be provided to the stimulation element at an appropriate timing, level, pattern, and/or frequency to achieve the desired function. However, the invention also contemplates eliminating the sensor in favor of applying a predetermined pattern of stimulation to the patient.

Apparatus and method for mitigating sleep and other disorders through electromuscular stimulation (U.S. Pat. No. 3,732,087). This electromuscular stimulator exerts a beneficial medical purpose selected from the group consisting of mitigating snoring, mitigating obstructive sleep apnea, mitigating hypertension, dental analgesia, general analgesia, monitoring physiological conditions and facilitating the intra-oral delivery of medication which is disclosed. The electromuscular stimulator includes a first electrode for making electrical contact with a first anatomical structure selected from the group consisting of a hard palate, a soft palate and a pharynx; a second electrode for making electrical: contact with a second anatomical structure; a control unit operably connected to the first and second electrodes; and a means for positioning the first and second electrodes relative to the first and second anatomical structures, respectively.

Dysphagia

U.S. Pat. No. 5,891,185. Said patent describes “a simple, non-invasive device and method for treating oropharyngeal disorders” provides electrical stimulation to the pharyngeal region of a patient. Oropharyngeal disorders may cause an inability to swallow or difficulty in swallowing.

Oral Devices and Methods for Controlled Drug Release

Oral Devices and Methods for Controlled Drug Release (PCT/IL2004/000123 dated 8 Feb. 2004). A controlled-drug-delivery oral device is implanted or inserted into an oral cavity, built onto a prosthetic tooth crown, a denture plate, braces, a dental implant, or the like. The device is refilled or replaced as needed. The controlled drug delivery may be passive, based on a dosage form, or electro-mechanically controlled, for a high-precision, intelligent, drug delivery.

Pulse Oximeter Sensor

Pacifier pulse oximeter sensor (U.S. Pat. No. 6,470,200). This pacifier pulse oximeter sensor includes pulse oximeter sensor elements located within the nipple of a pacifier. The poise oximeter sensor elements may be completely within the nipple material, embedded within the nipple material, nested within the nipple material, or adjacent so the nipple material while not being exposed to the outside environment. The pulse oximeter sensor elements include a light source and a light detector. The pulse oximeter sensor elements communicate with an oximeter through wiring, an electrical connector, and/or wirelessly. An alternative embodiment odds oximeter processing capabilities to the pacifier pulse oximeter sensor.

Intra-Oral Jig for Optical Measurement

Intra-oral jig for optical measurement (U.S. Pat. No. 6,430,422). A jig body of rosin is formed with a concave part engaging with an upper backside of teeth and another concave part engaging with a lower backside of teeth, and includes a portion coming into contact with an oral cavity part. An optical fiber bundle for measurement is embedded in the jig body, and a forward-end-surface of the optical rider handle is exposed on the portion of the jig body coming into contact with the oral cavity part and flush with the portion. A heater and a temperature sensor for keeping the temperature of the jig body constant as well as a pressure sensor for detecting a pressure for holding the Jig body between the upper and lower tooth of a measured person are further embedded in the jig body.

Method for Monitoring Arterial Oxygen Saturation

Method for monitoring arterial oxygen saturation (U.S. Pat. No. 6,263,223). Thus is a method for taking reflectance oximeter readings within the nasal cavity and oral cavity and down through die posterior pharynx. The method utilizes a reflectance pulse oximeter sensor that preferably is resistant to bodily fluids to contact one of these capillary beds for the taking of readings and then forwarding of these readings to an oximeter for display. The method includes inserting a reflectance pulse oximeter sensor into a cavity within a subject's skull and contacting a capillary bed disposed in the cavity wild the reflectance pulse oximeter sensor.

Intra-Oral Jaw Tracking Device

Intra-oral jaw tracking device (U.S. Pat. No. 5,989,023). A jaw tracking device, which fits entirety in the mouth and can be attached to conventional removable dental appliances tracks the location and movement of the lower jaw with high precision and speed when the mouth is closed or nearly closed by recording the projection of light from a light emitting diode, laser diode, or fiber-optic source fixed to the tower dental arch onto one or two position sensitive detectors (PSDS) fixed to the upper dental arch. Since the system acquires data quickly enough to record the minute deflections of the lower jawbone which occur each time the jaw is closed eccentrically, it can be used with acoustic sensors attached to the individual teeth in order to analyse a person's bite. Since each PSD relies on only four outputs, its data can be easily transmitted by telemetry so that it can be used to track the location of the jaw during sleep without requiring wires protruding from the mourn of the sleeping subject.

Intra-Oral Sensing Device (U.S. Pat. No. 4,629,424)

The appliance contains a number of sensors to monitor the parameter of interest and a telemetry unit plus power pack for signal transmission

Intra-oral sensing device to be placed into the mouth of a patient for producing tooth and jaw images (U.S. Pat. No. 5,691,539). An intra-oral sensing device for producing tooth and jaw images of a patient has a housing with a back. The housing has an interior. An image sensor is positioned in the interior of the housing. A printed circuit board with electrical contacts is positioned in the interior of the housing and connected to the image sensor. An electric cable, for connecting the sensing device to an image processing unit, is provided, it extends into the interior of the housing at a location of entry and has electrical leads. The electrical loads are connected to the electrical contacts of the printed circuit hoard. The electric cable extends from the location of entry at the housing at an angle of 0° to 10° relative to the hack.

Intra-oral sensor (U.S. Pat. No. 6,652,141). A new and improved intra-oral sensor for use in a filmless radiography system is disclosed. The sensor is configured to fit comfortably and close to a target area in an intra-oral cavity. By providing a comfortable relative fit to the target area, the sensor is ergonomically improved, in terms of its comfort and feel to a dental patient. In addition, the configuration of the sensor is designed to allow the sensor to be placed closer to a target area in an oral cavity than prior sensors (i.e. closer to target teeth, gum, etc). Moreover, the sensor is configured so that it can easily be located in a correct position relative to the target area, and when located correctly to properly position its sensing structure for receiving radiant energy. These features are believed to reduce refractive error in the image received by the sensor, thereby improving the Image data transmitted by the sensor.

Protecting electronic-based, medical devices, components and modules

In order to function properly and safety, components that are not intrinsically biocompatible must be protectively coated in a manner that does not adversely or Significantly affect mechanical tolerances, electrical characteristics or other critical performance characteristics. Furthermore, piecing electronic devices, components or circuits in a humid or wet environment requires protection of the electronic components by Isolating them from the surrounding environment, to prevent shocking of the electronic circuitry by the ions present in the oral liquids (saliva and intake liquids), corrosion and the development of bacteria, all are factors that may cause the device to malfunction within a relatively short time. The presence of a battery and DC current intensify the problem by generating concentrated corrosive activity in one direction. Electro-optical devices, operating on receiving or transmitting lights (intra Red or in the visible range) demand a transparent protection to allow light pass through the protective cover. Radio Frequency based communication, techniques require permeability to electromagnetic waves in uni- or bi-direction, while maintaining the RFI and EMI applicable standards. Furthermore, protective coating of a biomedical surface may be required for a number of reasons, including physical isolation from moisture, chemicals, bacteria, plague and other substances; surface passivation; electrical insulation, tie-down of microscopic particles; and reduction of friction.

Some of the more common protection methods are:
Encapsulation in metal case—In devices such as heart pacemakers or vagus pacemakers the electronic modules and batteries are encapsulated inside a metal case, usually made of titanium or stainless steel.
Conformal coating—Traditional conformal coatings are solvent-based liquid mains sued as epoxies, silicones, acrylics, and urethanes. Some liquid coatings are also available in a 100%-solid form without solvents. However, such materials sometimes exhibit liquid properties (pooling, meniscus, etc.) that may make them unsuitable for some medical coating applications, in addition, liquid coatings may not meet toxicity or biocompatibility requirements, and cannot, be applied with precise process control.
Parylene coating—A crystal-clear, polycrystalline and amorphous linear polymer material currently used to protect a wide variety of mechanical devices. This vacuum-deposited polymer coating, transparent and flexible, meets the requirements of a USP Class VI and can be applied as a film in layers as thin as 1 μm to provide pinhole-free and conformal coating, even on complex surfaces. Parylene has three types type “N”, type “C”, and type “D” each one has unique characteristics.
Plasma Sputtering—A surface treatment often performed over the coated target poor to adhesion of parylene or other conformal coating.

The following table compares the coating techniques described herein

| PROPERTY | ACRYLIC | URETHANE | EPOXY | SILICONE | PARYLENE |
|---|---|---|---|---|---|
| Uniform, very thin, | G | G | G | G | E |
| conformal layer | | | | | |
| Low stress, pin-hole free | M | M | M | M | E |
| layer | | | | | |
| Dielectric properties | G | M | M | VG | E |
| Physical strength | G | VG | VG | M | E |
| Flexibility | M | VG | L | VG | VG |
| Wear and abrasion | M | VG | VG | L | E |
| resistance | | | | | |
| Thermal coefficient of | G | M | VG | L | E |
| expansion | | | | | |
| Water absorption | G | G | VG | M | E |
| Chemical, solvents, fungus | L | VG | VG | M | E |
| resistance | | | | | |
| Barrier to moisture, gases, | VG | G | VG | M | E |
| liquids | | | | | |
| Adhesion to substrates | VG | G | VG | M | G |
| Repairability | VG | G | L | M | G |
| No contaminating | G | G | G | L | E |
| ingredients | | | | | |
| Particles immobilization | L | L | L | L | E |

G—Good;
L—Low;
E—Excellent;
VG—Very Good
M—Medium

Common Tests & Updates Performed Over Electronically Based Equipment During the Manufacturing Phase (i.e. Post the Development Phase)

Operability Tests

Functionality of the intra-oral devices is tested by emulating input signals and data to ensure mat the proper output and operation occurs without errors. Specific tests can include simulated and virtual inputs identifiers and virtual outputs handling and verifying proper alarm generations and responses.

Application Feature/Functional Testing

Feature testing is used to verify individual commands and capabilities of the application. Feature testing is also performed with multiple inputs to measure the interface and application operations or transactions invoked by the client. Functional testing shall be used to verify that the application's multi-characteristics and background functions work correctly under various scenarios and heavy loads Functional testing shall be performed under loading that closely models the substation's real-world operating environment.

Reliability Testing

Reliability tests are run under medium to heavy load to monitor the device errors and failures. Reliability testing forces e.g. the DUT (Device Under Test) or the communication to handle in a compressed time period the activity, it would normally experience over weeks, months, or years on a patients intraoral environment. Reliability testing attempts are made to accelerate feature of the processes or other devices caused by usage various patterns:

Boundary-Scan Testing and In-System Programming (ISP) Solutions

Boundary-Scan Testing was developed in the mid-1980s as the JTAG interface to solve physical access problems on PCBs caused by increasingly crowded assemblies due to novel packaging technologies. Boundary-scan embeds test circuitry at chip level to form a complete board-level test protocol. With boundary-scan (industry standard IEEE 1149.1 since 1980) one can access even the most complex assemblies for testing, debugging and in-system device programming end for diagnosing hardware problems.

Embedded Firmware Upgrade and Personal Profile Loading

Due to bug-fixing, updating of the requirement, new software (or firmware) embedded inside the intra-oral device has to be loaded, replacing the existing one. Furthermore, personalization of the device requires programming the device to match the patient's specific characteristics such as: medical status, age, weight, gender, origin. DMA is an option existing in few intra-oral devices.

### SUMMARY OF INVENTION

The present invention teaches a manufacturing method for intra-oral devices, customized devices and homogenous devices to be used in humans and in animals

It also teaches testing methods and devices for testing (testers) of intra-oral devices for applications such as salivary gland electro-stimulation, controlled drug delivery, bio-sensing of biological conditions, treating apnea, other sleeping disorders, eating disorders and neurological disorders by electro-stimulation.

The present invention thus consists substantially in a manufacturing method of en intra-oral device, to be used in humans as well as animals, which has an electronic module; characterized in that the electronic module is embedded in the device which is made of a bio-compatible material; having at least one exposed opening.

Electronic module in accordance with the present invention may be selected, however is not limited to: IC [integrated circuit], ASIC (application Specific IC), resistor, capacitor, coil, antenna, PCB [printed circuit board], diode, switch, photo-electric device, battery, power source, or combinations thereof, etc.

The openings in accordance with: the present invention may be selected however are not limited to: opening[s] for electrodes, opening[s] for the exit of medicine, opening[s] for the entrance of certain materials or combinations thereof, etc.

The manufacturing method should advantageously comprise the following steps:
i. placing an electronic module and/or a power source inside one or more layers and/or casting made of materials such as vinyl, silicone, acrylate, ceramic, polymers, metal, metal alloys or other dental material, or any combination thereof, in such a way that the electronic modulo and/or the power source remain embedded:
ii. protruding at least one pair of electrodes (when needed) or at least one opening for drug release (when needed) or at least one opening for oral fluids ingress (when needed) or at least one opening for the analyte egress (when needed) out of the first layer;
iii. covering the transceiver, (being a combination of a receiver and a transmitter) with an IR transparent material or an RF permeable material or any combination thereof; and
iv. testing the assembled apparatus functionality.

The manufacturing method of the electrodes if present should advantageously comprise the following steps
i. Manufacturing the electrodes out of a bio-compatible material such as NiTiNol (which stands for—Nickel (Ni), Titanium (Ti) and Naval Ordnance Laboratory (NOL)) or its alloy B, C, Dy70, Dy90, H, M, N, S, or stainless steel, or titanium or polymers with memory.
ii. The electrodes surface may be finished with electropolish, coated with polymers, plated with gold, silver, nickel, copper, titanium oxide or any combination thereof.
iii. Shaping the electrodes like an arc (FIG. 3b (70)), coming out of a plate (FIG. 3b (71)). Once inserted it fits itself to the patient's unique geometry of the mouth at the touching area of the electrodes and the human tissue. Once extracted the electrodes return to their original shape, utilizing the memory’ effect the material has,
iv. Connecting the electrodes using crimping or soldering method to the stimulating electronic circuit (FIG. 3b (72)).

By this method can be manufactured at least an intra-oral salivary gland electro-stimulator, an intra-oral controlled drug delivery device, an intra-oral device to draw biological analyte of interest specimens from oral tissues, for analyses inside or outside the intra-oral device, and an intra-oral device to treat phenomena such as apnea, sleeping disorders, oropharyngeal dysphagia eating disorders, neurological disorders by means of electro-stimulation.

In cases where Hie manufacturing method used is for the preparation or an intra-oral salivary gland electro-stimulator or an intra-oral device to treat phenomena such as apnea, sleeping disorders, eating disorders and neurological disorders by means of electro-stimulation; as step ii. “at least one pair of electrodes out of the fits; layer is used”, and one or more of the following additional steps may be performed:
i. masking the stimulating electrodes before final coating;
ii. coating the electronic module and/or power source with a protective coating such as parylene, a conformal coating, such as silicone, anti-bacterial coating, dental resins or any combination thereof prior to embedding it between the layers;
iii. encapsulating the electronic module and/or power source with a case made of metal such as titanium, NiTiNol, stainless steel, plastic material such as PVC, polymer: or any combination thereof prior to embedding it between the two sheets: and
iv. protruding the stimulating electrodes out of the box.

The manufacturing process may be used also for the manufacture of an intra-oral controlled drug delivery. In this instance one should use for step ii. “protrude at least one opening for drug release”. In this case the following additional steps should be performed:
i: coating the electronic module and/or power source with a protective coating such as parylene, conformal coating, such as silicone, anti-bacterial coating, dental resins or any combination thereof prior to embedding it between the layers;
ii. connecting to the above drugs reservoirs to the above intra-oral device; and
iii. protruding at least one opening for drug release.

The manufacturing process may be used also for the manufacture of an intra-oral device which draws biological analytes of interest specimens from oral tissues to the mucosal surface, for analysis inside or outside the intra-oral device, in this instance one should use for step ii. “protrude at least one opening for analyte ingress”. In this case the following additional steps should be performed:
i. coating the electronic module and/or power source with protective coating such as parylene, confer mat coating, such as silicone, anti-bacterial coating, dental resins or any combination thereof prior to embedding it between the layers;
ii. protruding at least one opening for analyte ingress; and
iii. connecting to the specimen reservoirs to said intra-oral device.

The manufacturing and assembly methods of intra-oral devices manufactured by the present invention are described hereinafter.

Customized and Non-Customized Assembly and Manufacturing Methods

A device placed inside the oral cavity should be adopted to match the individual anatomy or be designed in a generic manner to match the majority of the users. The present invention teaches the manufacturing methods that produce customized devices or generic versions thereof that fit all the devices described in the present invention.

Customized

The major slops of the customized devises manufacturing processes are;
1. the patient's dentition and oral cavity impression are taken with polyvinylsiloxane, alginate or similar materials or by a 3 dimension electronic scanning (LASER based or similar);
2. the physical impression or the files describing the individual oral cavity is shipped to the manufacturing laboratory or the field generated by electronic scanning is sent to the manufacturing laboratory using an electronic media (disc. CD, NY memory or via the internet;
3. based on the impression a laboratory manufactures a model that matches the individual impression (similar to the manufacturing of a standard mouth-guard or dental denture);
4. an outer layer of the material in use in the dental industry such as vinyl, polymers, acrylate, silicone or other dental or bio-compatible grade material, is applied on the device-bearing surfaces of the module, which are similar to those used for a regular mouth-guard or night-guard;
5. then, the non-customized elements such as the electronic module, battery, drug delivery device, drug reservoir, sensors are placed, in the most, appropriate location, i.e. where the interference to the user is minimal and the effectiveness is maximal;
6. then, those non-customized elements of step 5 are coated by a layer of material similar to that of step 4. The result is a ‘sandwich’ like configuration where the non-customized elements are embedded between coating dental materials; and
7. finally, one (or more) of the external interfaces are set to allow:
   a. egress of drugs and fluids in the drug delivery devices;
   b. ingress of oral fluids, such as saliva, blood for bio-sensors and monitoring devices;
   c. protrusion of the stimulating electrodes to electro-stimulate the muscles and nerves in the electro-stimulator applications such as the salivary glands stimulator, treatment for snoring and apnea, eating disorder, obesity and dysphagia;
   d. optical communication is performed with the intra-oral device via a transparent coating in the visible, intra Red (IR) or ultraviolet spectrum; and
   e. RF communication to pass to/from the device coating allowing communication with the device.

Non-Customized

The non-customized device can Pave tour basic designs a) a tooth like device, b) a demure like design and c) a clip hooked to a teeth or artificial implant d) and a soft tissue (such as tongue, cheek, etc.) retractor;

The major steps of a tooth tike non-customized devices manufacturing processes are:
a. a module should carry and embed the non-customized elements, such as the electronic module, battery, drug delivery device, drug reservoir, sensors. Those elements are coated by a material in use in the dental industry such as vinyl, acrylate, silicone or other dental or bio-compatible grade material; and
b. protecting and sealing of the elements that need to be protected such as the electronic elements, battery, sensors, and communication elements.
c. external interfaces for the non-customized persons are similar to those which are set out above for the customized persons.

The major steps of a denture like and soft tissue retractor non-customized device manufacturing processes are:
a. a module or one or few sizes (usually small, medium and large) molds, similar to the one used by sportsmen to protect their teeth is used instead of the customized mold to carry and embed the non-customized elements, such as the electronic module, battery, drug delivery device, drug reservoir, sensors. Those elements are coated by a material in use in the dental industry such as vinyl, acrylate, silicone or other dental or bio-compatible grade material;
b. protecting and sealing of the elements that need to be protected such as the electronic modules, batteries, sensors, and communication elements; and
c. the user can (in those devices) adjust the device by a method known as “boll and bite” which the device is warmed (by hot water as an example; and the user bites the device to imprint one's specific anatomic topography on the device shape; and
d. external interfaces for the non-customized persons are similar to those which am set out shove for the customized persons.

Protection Methods Over the Electronic Elements are Described Hereinafter

The oral cavity exhibits a very harsh environment to embedded electronic elements and to power sources. Characterised by high temperature (of 37 degrees centigrade), constantly wet, rich with large variety of chemical compounds; small ions, positive charged, negative charged, low pH (such as Coca cola of ˜2 pH), high pH (lemon juice etc.), mastication forces applied and constant movement of the jaws and more, in order to protect electronic devices (integrated Circuits (IC), circuits, printed circuits, passive elements, optical elements, ewe a protection method has to be applied through the manufacturing process to guarantee lasting of the electronic element over time within the oral cavity. The present invention teaches the use of one (or more) of the following manufacturing methods:
1) Activating the surface of the sensitive elements using a method known as ‘sputtering’ or ‘Plasma sputtering’ prior to coating with protective coating material such as Parylene or adhesives;
2) Coating hie sensitive elements with Parylene prior to embedding as described in the following steps;
3) Embedding the sensitive elements inside layers of silicon, acrylic, polymer, metal foil or any combination thereof, a two layers ‘sandwich’ like or coating mold;
4) Using bio-compatible materials for embedding the sensitive elements inside the silicon, acrylic, polymer, metal foil or any combination thereof; and
5) Sealing with bio-compatible glue the holes.

Manufacturing stages according to the present invention are advantageously sub-divided into two major branches a) at the dental clinic b) at the manufacturer:

At the Dental/Physician Clinic
An individual mouth impression is taken from the patient's mouth.
The impression is sent to the manufacturing center or a computerized 3D scanned model of Hie impression is sent to the manufacturing center.
Upon receiving the assembled device, the patient is instructed and begins its use.

At the Electronic Manufacturer
The electronic components and battery for batteries) are assembled on a flexible (or rigid) PCB (23) (Printed Circuit Board) of FIG. 1.
The assembled PCB is cleaned from dust.
The assembled PCB is Gleaned by decreasing solvents.
The assembled PCB is tested for Us proper functionality.
The assembled PCB is treated with plasma sputtering or silane such as A174, or combination thereof.
The assembled PCB: including the battery but excluding the stimulating electrodes, ara coated with 5-25 μm thick layer of parylene.
The assembled and coated PCB functionality is tested.
The assembled PCB is switched to power saving mode, until initial usage is started.

At the Final Assembly and Test Site Using Vinyl or Acrylate or Other Dental Material the Following Steps May be Performed
A dental technician makes a regular dental plaster mouth model from the patient's impression.
Over the model, a vinyl, acrylate, silicone, polymer or other dental material sheet is laid, covering partially or fully the dental arch.
The assembled PCB is placed over the first vinyl, silicone, acrylate, polymer or other dental material layer; the stimulating electrodes protrude out of this first layer facing the jaw (preferably in the lower third molar area, lingual side)
An additional vinyl, acrylate, silicone, polymer or other dental material layer is placed on top of the first layer covering the PCB and battery.
The layers are adhered and melted together.
The device is tested for its proper functionality.
The device is set to power saving mode,
The device is sent to the dental clinic.

The manufacturing method described herein is also suitable for the preparation, e.g. of an apparatus which stimulates the salivary glands which apparatus comprises:
i. a mouthpiece suitable to detachably engage teeth and an appliance including;
ii. a socket designed to cover at least one tooth;
iii. an electrical stimulator Circuit associated with the socket, whom the electrical simulator produces electrical pulses when actuated;
iv. a power source unit; and
v. a receiver including a receiver module and a decoding circuit for remote control.

Said apparatus may also comprise one or more of the additional following features:
i. a wetness sensor unit designed to sense the intraoral wetness level;
ii. the commands received from the intra Red receiver, RF receiver or any combination thereof select the desired electro-stimulation level out of pre-defined stimulation patterns;
iii. the wetness level received from the wetness sensor selects the desired electro-stimulation level out of pre-defined stimulation patterns;
iv. the transmitter unit from the mouthpiece includes a Light Emitting Diode (LED), RF transmitter or any combination thereof; and
v. the receiver unit of the mouthpiece includes art intra Red photodetector and receiver modules, the wireless Radio Frequency (RF) based transceiver, directly contacts a control or any combination thereof.

A similar apparatus which stimulates the salivary glands and includes the same parts as the previous apparatus described above wherein said appliance is a customized custom-made appliance and does not comprise a transceiver may be prepared.

Said apparatus includes at least one electronic module as described in FIGS. 1, 1a and 1b (an ASIC (25), or a microprocessor (25), etc) and at least one power source such as battery (24), incorporated into at least one tooth socket or region thereof. The region is selected so that the stimulating electrodes (21) will be most effective, preferably near the lower third molar. The embedded electronic produces electrical signals at pre-defined patterns, voltage and currents applied on the oral tissue where if is most effective, preferably lingually to the lower third molar site.

The circuitry is preferably designed to produce an stimulating signal output of between 1 μA to 1 mA, preferably 10 μA to 500 μA, more preferably 20-260 μA, most preferably 50-150 μA. According to a preferred embodiment of the present invention the signal generator includes a mechanism for producing a series of pulses having an amplitude of about half to ten, preferably one to eight, more preferably two to four Volts, a pulse width of about 1-10000, preferably about 300-2000, more preferably about 1000 μseconds and a frequency of about 1-160, preferably about 2-50, more preferably, about 5-20 Hz. The occult is preferably designed to produce uni-polar or bi-polar pulse, more preferably bi-polar pulses

The number of simulating electrodes is preferably ten, more preferably four, moss preferably two. The distance between the electrode pair is preferably 2-10 mm more preferably 4.5-6.5 mm. The electrodes are made of metal such as platinum, stainless steel, gold, aluminum, copper, metal alloy.

The present invention also provides a removable oral appliance coupled void a transceiver {22} as described in FIGS. 1, 1a and 1b to receive and transmit the control signals from a remote control unit, by using intra red (40), such as in the normally used home appliances, or RF antenna and circuit. The remote control is able to increase (41) and decrease (42) stimulus intensity by changing parameters such as amperage, voltage, frequency and duly cycle, increase or decrease drug dosage level, increase or decrease measurement frequency, and to present the stimulation level, drug amount remains inside the intraoral reservoir and the results of the bio-sensing, in both numeric and Alfa-numeric characters, as described in FIGS. 3 and 3a.

The present invention also provides a removable oral appliance coupled with a power source induced or direct, preferably two batteries mom preferably one battery (24) of FIGS. 1a and 1b, preferably secondary (rechargeable battery) more preferably primary battery, preferably producing voltages 1.2 V-9V more preferably 1.5V-6V, more preferably 3V-4.5V.

The remote control uses a protocol such as Manchester code, Philips RC5, to send and receive data to/from the intra-oral device. It has few control buttons preferably 25 more preferably 14, more preferably 2.

Testing Methods & Testing Techniques of Intra-Oral Devices

Testing in this connection consists of three major elements: a) a Device Under Test (DUT), which includes the device to be placed intra-orally and its accessories, b) a Testing Apparatus—TA and c) a testing script, test programs and instructions that produce a series of predefined scenarios of inputs and simulates the environment while measuring the output.

The simulated input simulates various conditions and tests the proper functionality of the DUT under those conditions. More specifically the input can be in the form such as an electrical signal wireless commands, simulating the personal remote control, wetness, simulating the saliva flow, noise (simulating snoring etc.), electrical noise, biological substances (such as glucose level, lactate, INR, BNP), flow rate (drug low rate and quantity), or any combination thereof. Upon completion of the tests each output is compared against the expected result and a specific algorithm defines for each test whether it ‘Passed’ or ‘Failed’. The tests results are presented to the operator in the form of electronic notice such as a display on a monitor or a paper printout. The tester may be connected to additional testing equipment such as standard laboratory equipment (Digital Volt Meter, oscilloscope, current meter, noise meter, etc.), computers (such as a PC (50) shown in FIG. 4, PDA (32), mainframe shown in FIGS. 2 and 4) or any combination thereof.

The present invention also consists in a method for testing an intra-oral device, to be used in humans as well as animals, which has an electronic module; characterized in that the electronic module is embedded in the device which is made of a bio-compatible material; having at least one at least one exposed opening.

Said method may be used inter alia for testing; an intra-oral salivary gland electro-stimulating device; an intra-oral controlled drug delivery device; an intra-oral device for the measurements of blood, oral fluids, other analytes of interest or any combination and an intra-oral device to treat apnea, snoring, sleeping disorders, eating disorder, oropharyngeal dysphagia neurological disorders.

The intra oral device having an electronic module may be tested by the following method which comprises the steps of:
i. connecting the Device Under Test (DUT) to a tester;
ii. applying a combination of inputs signals and parameters to the DUT in accordance to a pre-defined scripts;
iii. measuring the functionality of the tested intra oral device and comparing it to pre-defined expected results;
iv. Applying pre-defined criterions for ‘pass’ or ‘fail’; and
v. reporting test results.

The above method may comprise the following additional feature:

connecting the DUT to laboratory equipment such as digital volt meter, oscilloscope, flow meter, PC analyzer or any combination thereof.

This method is advantageously performed after manufacturing, before clinical use, at the operation theater, at the clinician clinic or any combination thereof.

Testing Methods

The device external interfaces and test points are connected to the tester. Wireless communication elements (Infra Red or Radio Frequency) are placed within an effective communication distance from the DUT. Sockets are ready to receive an intra-oral device, while the socket on the left carries such a device. The test scripts include measurements and test that assure the proper functionality of the DUT. The script may include few (or all) of the following tests:

Tests procedures are activated, the tests procedures can include (but are not limited to):
1. Measuring DUT built-in battery voltage;
2. Measuring DUT built-in battery max drain current;
3. Measuring DUT inputs impedance;
4. Measuring DUT output impedance;
5. Measuring DUT output max current source/sink;
6. Pleasuring wireless communication sensitivity;
7. Pleasuring sensors functionality and accuracy;
8. Applying a pre-planned scenarios (52, 61) shown in FIG. 4, including, stimulating the DUT inputs and measuring DUT output and comparing them against expected results,
9. Applying several scenarios, simulating real situations, extreme input conditions such as multiple, simultaneous input, varying environmental situations, such as high and low temperature, humid and wet;
10. Measuring membrane permeability;
11. Pleasuring drug delivery mechanism; and
12. Measuring sensors functionality and accuracy.

Upon completion, a test report, indicating Pass or Fall is produced.

An optional log file; specifying the performed test, and Pass/fall indication per test, recommended action and failure description may be produced as an electronic report of print out on paper.

Device Under Test (DUT Configuration)

DUT configurable parameters are programmed to match specific needs: such needs are, e.g. selecting the stimulating electrodes active pair (in salivary glands electrical simulator), communication type and speed, patient's specific drug delivery pattern to match his/her personal profile such as; weight, gender, ace, DNA profile, medical history, origin.

The configurable parameters may be stored in a nonvolatile memory or battery backup memory.

Testing Apparatus (TA) Configuration

The intra oral device having an electronic module may be tested by a tester apparatus, to be used in humans as well as animals, which has an electronic module; characterized in that the electronic module is embedded in the device which is made of a bio-compatible material; having at least one exposed opening.

In said tester the intra oral device may be selected among intra-oral salivary gland electro-stimulating device; an intra-oral controlled drug delivery device; an intra-oral device for the measurements of blood, oral fluids, other analytes of interest or any combination and an intra-oral device to treat apnea, snoring, sleeping disorders, eating disorder, oropharyngeal dysphagia neurological disorders.

There may be inter alia the following testers.

### Tester A

An electro-stimulator tester (as indicated above) comprising:
i. an interface to the Device Under Test (DUT);
ii. a state machine;
iii. a testing script; and
iv. input and output devices.

In said tester the state machine may be selected among a microprocessor, an Application Specific IC (ASIC), an electronic module based on off the shelf discrete electronics components or a personal computer.

The tester may be connected to e.g. a PC (Personal computer) based on RS232, USB, wireless LAN, Bluetooth, WiFi, intra Red, proprietary bus or any combination thereof; or

to a PDA (Personal Digital Assistant) based on USB, wireless LAN, Bluetooth, Zig-Bee, WiFi, Intra Red, proprietary bus or any combination thereof.

The above tester may comprise in addition one or more of the following features.
a. an intermediate, detachable receptacle for the DOT placed at sockets (37) which connects the DUT and the Test Apparatus (FIG. 2b), allowing sterilization, cleaning, wiping and any combination thereof of the receptacle unit:
b. an additional testing script being based on pre-defined input sequences and comparing the output to the expected results;
c. a tester which measures one or more of the parameters such as the DUT built-in battery voltage, measuring DUT built-in battery max drain current, measuring DUT inputs Impedance, measuring DUT output impedance, measuring DUT output max current source/sink, measuring wireless communication sensitivity, varying environmental situations such as high and low temperature, humid and wet or any combination thereof;
d. up-grading of DUT software or firmware or database being made through the connection wired or wireless to the tester unit;
e. programming the parameters, such as selecting the stimulating electrodes active pair, communication type and speed, patients specific stimulation pattern, stimulation strength, stimulation voltage, stimulation current, to match his/her personal profile including health history, health status, DNA profile, gender, ace, weight or any combination.
f. These configurable parameters are preferably stored in a nonvolatile memory or battery backup memory, and finally
g. the tester reports results, indicating ‘Pass’ or ‘Fail’. A log file specifying the performed test, and Pass/fail indication per test, recommended action and failure description, or any combination thereof, may be produced as art electronic report or print out on paper.

The following testers may be used inter alia in addition to the salivary gland electro-stimulator tester;
B. a tester for an apparatus of intra-oral, controlled drug delivery device;
C. a tester for an apparatus of intra-oral sensor of biological parameters such as glucose level, blood pressure, heart rate, blood oxidation, nitric oxide, lactate, hemoglobin, blood cells and platelets, triglycerides, cholesterol. INR, BNP, lactate, temperature, pH, pulse, pCO2, pO2, metals, such as copper, cadmium markers of cardiac injury, such as troponins T and I, ischemia-modified albumin, fatty acid-binding protein, drugs, such as lithium, naltrexone, or any combination thereof; and
D. a tester for an intra-oral electro-muscular stimulation device to treat breathing disorders, snoring, apnea, eating disorders, oropharyngeal dysphagia neurological disorders or any combination thereof.

Said testers B to D may be constructed substantially by the same parts as indicated for the salivary gland (see A above). As to the additional parts:

### Tester B

Tester B may comprise one of the following features:
1. a detachable receptacle for the DUT output which connects the DUT and the Test Apparatus;
2. measuring parts of the tester which measure in addition DUT drug output, measuring DUT drug output minimum level, measuring DUT drug output maximum level, measuring DUT drug level sensor accuracy and resolution, measuring DUT drug flow rate sensor accuracy and resolution:
3. the following additional parameters are re-programmed drug delivery pattern, drug delivery schedule, patient's specific drug delivery pattern; and
4. sensors measure the pattern of drug releases during the test session.

### Tester C

Tester C may comprise one of the following features:
1. measuring parts of the tester which measure in addition sensed parameter sensitivity, sensed parameter accuracy, sensed parameter resolution, sensed parameter tolerance to external interferences:
2. re-programming the following additional parameters, e.g. selecting the measured biological analyte, patient's specific stimulation delivery pattern; and
3. sensors measuring the amount of accuracy of the DUT sensing during the test session and measure the amount of measurement resolution of the DUT sensing during the test session.

### Tester D

Tester D may comprise one of the following features:
1. measuring pads of the tester which measure in addition stimulation sequence and delays between the different probes, measuring stimulation sequence and delays between the different probes; and
2. re-programming the following additional parameters selecting the active stimulating electrodes, patient's specific stimulation pattern, stimulation strength, stimulation voltage, stimulation current.

The Test Apparatus (TA) is composed by two major elements a) Testing Apparatus adaptor (TAA) serving as a mediator between the TA and the accessories such as the PC or PDA b) Testing apparatus accessories such as a PC or a hand held computer (known also as Personal Digital Assistance or PDA). The major building blocks of the TAA (55) are: DUT (36), DUT input/output interfaces (58), wireless interface (56), TA embedded processor or state machine (53), TA software 161), Testing scripts 152), power source (57).

The major building blocks of a PC cased TA are; DUT (36), DUT input/output interfaces f 58), wireless interface (56), TA embedded processor or state machine (53), TA software (51) Testing scripts (52), power source (57), PC (50), Software (60), Testing scripts (61), Interfaces (62).

As a subset, the TA functionality is null and the PC perform all its tasks.

The major building blocks of a PDA based TA are: DUT (38). DUT input/output interfaces (58), wireless interface (56) TA embedded processor or state machine (53), TA software (51), Testing scripts (52), TA state machine (53), power source (57), PDA (32), FDA Software (83), PDA Testing scripts (64), PDA and DUT/TAA interfaces (65).

All the above elements are described in FIGS. 2, 2a and 4.

The present invention will now be illustrated with reference to the following Examples and drawings but is not limited thereto.

### EXAMPLES

### Example 1

In this example, showing the efficacy of the salivary glands electro-stimulator, which was manufactured by a method mentioned herein, 96 experiments in 14 patients with dry mouth using the electro-stimulating device, where designee as follows;
1—Saliva was collected initially in a storing tube.
2—The device was woken-up using the testing apparatus.
3—The device was placed in the subjects' mouth for 10 minutes.
4—According to a certain schedule, a command via a remote control was given to the device to be activated to a certain stimulation pattern or not to provide any stimulation (placebo). Both, the patient and the operator had no knowledge about the selection between “active” and “Placebo” options.
5—After 10 minutes the device was removed and saliva was collected
6—After furthers minutes saliva was collected again.
7—After further 7 minutes saliva was collected again,
8—After a resting period of 20 minutes, saliva was collected again
9—The intra-oral device was placed in the subjects' mouth again for 10 minutes.
10—According to the schedule, a command via a remote control was given to the device to be activated to a certain stimulation pattern or not to provide any stimulation (placebo). Both, the patient and the operator were blinded to the schedule. At each such session either the first or the second lest was placebo fine distribution along the experiments of placebo given as the first or second test, was equal).
11—After 10 minutes the intra-oral was removed and saliva was collected.
12—After further 3 minutes saliva was collected again,
13—After further 7 minutes saliva was collected again,
14—Finally patients were asked which test had a defter effect.
15—Each saliva sample was weighed to determine its volume gravimetrically utilizing the fluid accumulated in the storing tube, assuming a specific weight of 1. Flows were expressed as mL/min.

After 55 experiments, a second set of 41 experiments, containing reordering of the stimulation patterns to avoid research bias, was done.

The results ere summarized in the following table;

| | | | % increase | Absolute increase |
|---|---|---|---|---|
| | | Mean | compared to | compared to |
| | | measured | secretion before | secretion before GN |
| Pattern | Timing | secretion | GN placement | placement |
| Placebo | Before device | 0.32 | | |
| | placement | | | |
| | Immediately after | 0.71 | 2.25 | 0.40 |
| | removal | | | |
| | 3 min after removal | 0.40 | 1.25 | 0.08 |
| | 10 min after removal | 0.32 | 1.01 | 0.00 |
| Active | Before device | 0.34 | | |
| | placement | | | |
| | Immediately after | 0.66 | 1.94 | 0.32 |
| | removal | | | |
| | 3 min after removal | 0.46 | 1.36 | 0.12 |
| | 10 min after removal | 0.38 | 1.10 | 0.04 |

The difference between placebo and active at the last collection (10 min after) was high statistically significance (p=0.01). Patients expressed a clear preference for the active mode, as seen in the following table:

Patient's Report on their Subjective Feeling:
Placebo is preferred 6

### Equal 17

Active is preferred 22

The following conclusion can be drawn:
1—There is a statistically significant difference between placebo and active, in the salivary secretion measured 10 minutes after device removal (20 minutes time point).
2—There is a clear preference of the patients in favor of the active mode.
3—The immediate effects (until 3 minutes after device removal) are attributed to mechanical stimuli in the mouth.
4—After the 13 minutes time point, the mechanical effect disappears (as seen in the placebo arm, whom salivary secretion returns to a level similar to the baseline).
5—Only in the active arm, a residual stimulating effect persists (as seen in the still higher secretion rate at the 20-minutes time point compared to baseline).
6—This persistent effect is attributed to long-lasting effect of electro-stimulation of the active arm.

7—The salivary glands of dry mouth individuals have a good response to electro-stimulation.
8—A small increment in salivary secretion is enough to achieve patient satisfaction.

### Example 2

The testing unit for the intra-oral device is based on a PC or a PDA. The purpose of die unit is to perform wake-up, simulation and the electro-stimulation device testing. The product is con-posed by two main parts:

### Electronics

The electronic is centered on a microprocessor, designed for very low power consumption such as Texas instruments MSP430. Additional circuitry support a) IR receiving circuit based on a photo-diode (with receiving center frequency at 920 nm) and Operational amplifiers, b) wetness sensor which measures the saliva film thickness by measuring its electrical conductivity, c) two stimulating electrodes spaced at 6 mm apart, d) a single lithium coin cell battery, e) additional supporting security such as multiplexes, operational amplifiers capacitors resistors and coils. The entire operation is controlled by embedded software. The microprocessor built-in power saving modes are used to minimize the power consumption of the device extending the life time of the apparatus before replacing or recharging the battery or the apparatus.

### Package

The package is made of plastic by an injection method and is composed of two components: the receptacle for the intra-oral device and the receptacle for the PDA. The interaction between both receptacles is wireless using IR light. If a PC is used instead of a PDA, the receptacle for the PDA is not needed.

The intra-oral device receptacle includes a connection to a PC, DC entrance infrared connection. This component may interact with a PC with no need to use the connection to the PDA. Thus, it contains all the necessary electronic elements to function independently. At its back, a USB connector and DC entrance are found.

All the functions of the testing unit are performed through commands given to the PDA or the PC Embedded software enables the fulfillment of an the functions. The software is required to receive analog signals from the intra-oral device, convert them to digital signals and transmit the results through the USB connection and/or the IR LED.

### General Micro-Controller Qualities are:

1. The software is written for a Microchip® micro-controller.
2. The chosen micro-controller has an internal A/D with a minimum of 8 bits resolution. The infernal A/D has 8 multiplexed inputs
3. The software is written in C and some parts may also be written in assembly.
4. The C compiler is a licensed version of Hitachi C Compiler™.
5. Only the compiled cede is delivered to the ordered.

### USB Connection Specifications:

1. The device is able to communicate with USB devices both as a host and as a Client.
2. The USB adapter IC is Phillips ISP1362 or any other IC in the market that supports USB 2.0 specification.
3. The USB connection supports low-speed (1.5 Mbit/sec) data transfer.

### Example 3

### Showing the Manufacturing Process of a Typical Customised Electro-Stimulator

A dry mouth patient approaches his/her clinic seeking a solution for the disease. The clinician takes an impression of the patent's lower law and sends it to the manufacturing center. At the center the technician produces an oral appliance made of vinyl, encapsulate the electronic modules (including the battery) inside, and cover it with second layer of vinyl after protruding the electrodes to stick out of five lingual side, close to the location of the third molar. The entire device is tested and put into a low power saving mode, packed and shipped to the clinician. Upon receiving the device, the clinician test its proper functionality using the TA, including waking up the electronic and microprocessor, upgrade embedded software si needed, feed inside personal parameters (if needed) and provide it to the patient to be used at his/her convenience. The patient uses the remote control to set the stimulation level at the preferred level including no-stimulation state to minimize power drain.

### BRIEF DESCRIPTION OF THE DRAWINGS

With specific reference to the drawings in detail, it is stressed that the particulars shown are by way of example only and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in mom detail than is necessary for a fundamental understanding of the invention, the description taken with the drawings making apparent to those skilled in the ad how the several forms of the invention may be embodied in practice.

In the drawings: The parts appearing in said drawings are indicated by the numerals.

FIGS. 1, 1a, 1b schematically illustrates the salivary glands electro-stimulator device;

FIGS. 2, 2a schematically Illustrate the structure of a tester apparatus, as known;

FIG. 2b schematically illustrates tester apparatus receptacle interface for salivary glands electro-stimulator crown like version;

FIGS. 3, 3a schematically Illustrate the wireless remote control; FIG. 3b schematically Illustrates the structure of the stimulating electrodes and

FIG. 4 schematically illustrate the tester apparatus block diagram showing its major components and optional major components.

### DETAILED DESCRIPTION OF THE DRAWINGS

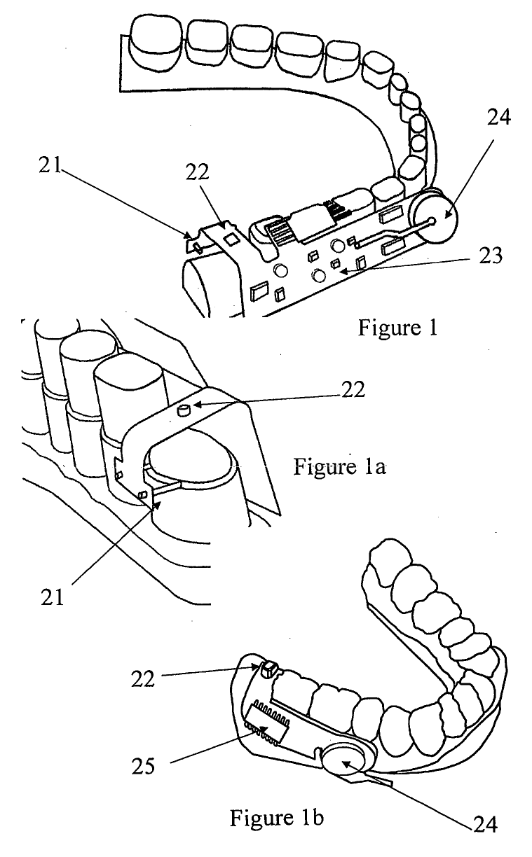

FIG. No. 1—Depicts an intra-oral device for the stimulating of the salivary glands as placed on lop of the lower law. The electronic module includes elements such as the PCB (23), the battery (24) the photo transceiver (22) and Pie stimulating electrodes placed next to the third molar, on the lingual side, (21). FIG. 1a shows the possible structure of the salivary glands electro-stimulator from a different viewing angle, i.e., the lingual side. FIG. 1b depicts the electronic module with a single IC control module (25) which can be a microprocessor, custom electronic or an ASIC.

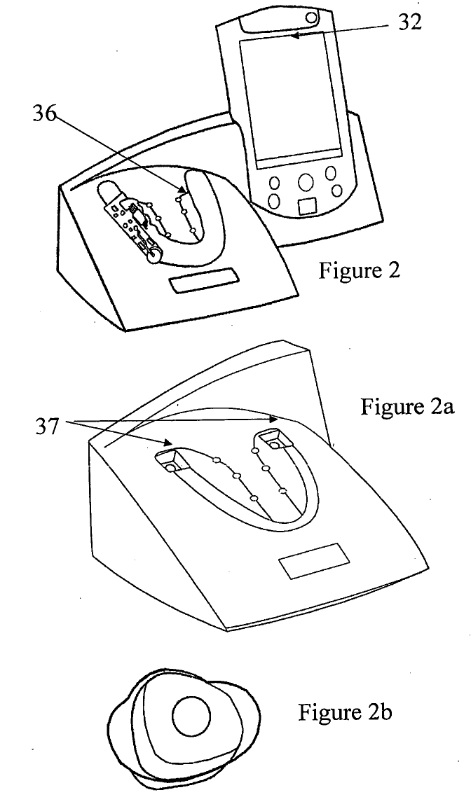

FIG. 2—depicts the Testing Apparatus (TA) which includes elements such as the Device under Test (DUT) (36) placed on top of the TA. PDA (32) controls the testing process and serves as an I/O device. FIG. 2a depicts the sockets for crown like intra-oral devices to be tested (DUT) and the DUT test interface (58) optional location. FIG. 2b depicts the socket which is an intermediate module between the crown like DUT and the TA, allowing easy cleaning and better hygiene.

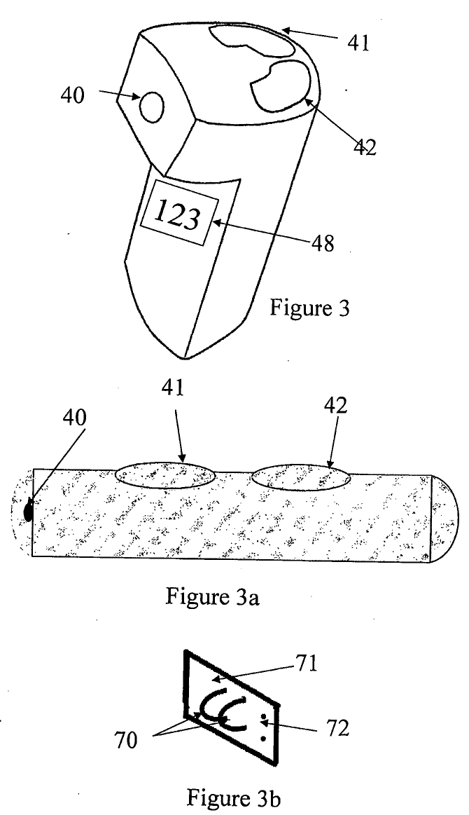

FIGS. 3 & 3a—depicts the remote control the patient can use to control the intra-mural device. It has two (as an example) buttons to increases or decrease buttons (41, 42) of the electro-stimulation signals, or drug dosage, and transceiver, like an IR LED transmitting signals and receiving IR signal (40). The remote control can come in two shapes: (a) like an inhaler and (b) like a pencil. Both are design in a user-friendly manner, in order to be used also by elderly people. The remote control can include also a display showing data and commands to the user (48), F-g 3b schematically Illustrates the structure of the stimulating electrodes (70) as placed on a PCB (71) and connected to the stimulating circuit (72).

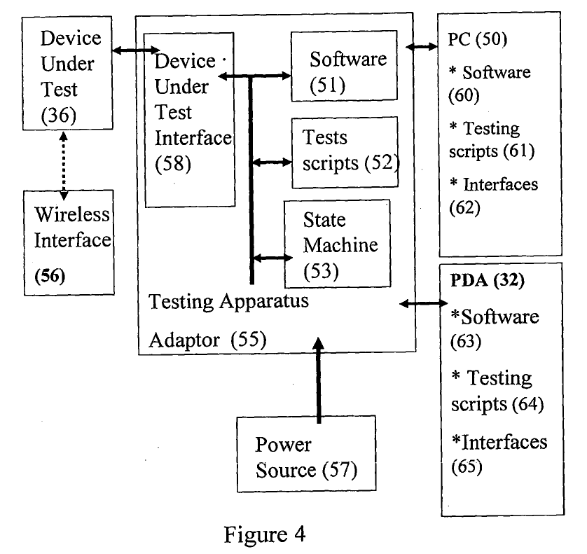

FIG. 4—depicts the block diagram of the various TA options. The three basic modules are: Device under test (36), which is the device to be tested, the control and I/O interface (a PC (50), PDA (32) or similar) and the testing scripts (64, 61). A power source (57) feeds the TA adaptor (55) and in some cases also the PC (50) or too PDA (32). Wireless control and test is done via the wireless interface (56).

1-36. (canceled)

37. A method of manufacturing and intra-oral device, to be used in humans as well as in animals, said intra-oral device made of a bio-compatible material and having at least one exposed opening, said method comprising the following steps:
i. placing an electronic module having a transceiver, inside one or more layers and/or a casting such that the electronic module remains embedded within said layers and/or casting;
ii. creating at least one opening in an outer layer of said one or more layers and/or in said casting;
iii. covering the transceiver with infrared (IR) transparent material or radio frequency (RF) permeable material; and
iv. testing the functionality of the intra oral device.

38. A manufacturing method according to claim 37 wherein the electronic module is an integrated circuit (IC), Application Specific IC (ASIC), resistor, capacitor, coil, antenna, printed circuit board (PCB), diode, switch, photo-electric device, battery, power source or any combination thereof.

39. A manufacturing method according to claim 37 wherein said at least one opening is an opening for electrodes, opening for the exit of drugs, opening for oral fluid ingress, opening for analyte ingress, or any combination thereof.

40. A manufacturing method according to claim 37 wherein said one or more layers and/or said casting are made of vinyl, silicone, acrylate, ceramic, polymers, metal, metal alloys, other dental material, or any combination thereof.

41. A manufacturing method according to claim 37, wherein said electronic module further comprises at least one pair of electrodes made of NiTiNol (Nickel (Ni), Titanium (Ti) and Navel Ordnance Laboratory (NOL)), stainless steel, titanium, polymer or other bio-compatible material and wherein said electrodes are shaped as an arc connected to a base plate and to an electro-stimulating circuit, and wherein said method further comprises the step of protruding said at least one pair of electrodes out of said at least one opening.

42. A manufacturing method according to claim 41 wherein the electrodes' surf ace is finished with electropolish, coated with polymers and/or plated with gold, silver, nickel, copper, titanium oxide or any combination thereof.

43. A manufacturing method according to claim 41 further comprising one or more of the following additional steps:
i. masking the stimulating electrodes before final coating;
ii. coating the electronic module with a protective coating, a conformal coating, anti-bacterial coating, dental resins or any combination thereof prior to embedding it between the layers and/or casting; and
iii. encapsulating the electronic module with a ease made of metal. NiTiNol, stainless steel, plastic material, polymer, or any combination thereof prior to embedding it between the layers and/or coating.

44. A manufacturing method according to claim 37 further comprising the following additional steps:
i. coating the electronic module with a protective coating, conformal costing, silicone, anti-bacterial coating, dental resins or any combination thereof prior to embedding it between the layers and/or casting; and
ii. connecting at least one drug reservoir to the above intra-oral device; wherein said at least one opening is structured and arranged to be used for drug release.

45. A manufacturing method according to claim 37, further comprising the following additional steps:
i. coating the electronic module with protective coating, conformal coating, dental resins or any combination thereof prior to embedding it between the layers;
ii. connecting at least one specimen reservoir to said intra-oral device, wherein said at least one opening is an opening for analyte ingress.

46. A manufacturing method according to claim 37 further comprising the steps of:
A. creating an impression of the patient's dentition and oral cavity with a dental impression material or by a 3 dimension electronic scanning;
B. transmitting the impression of the oral cavity to a manufacturing laboratory;
C. based on the impression, said laboratory manufactures a model that matches the impression;
D. applying an outer layer of vinyl, polymers, acrylate, silicone or other dental or bio-compatible grade material on device-bearing, surfaces of the module;
E. placing the electronic module, battery, drug delivery device, drug reservoir, and/or sensors on said model;
F. coating said electronic module with a layer of vinyl, polymers, acrylate, silicone or other dental or bio-compatible grade material such that said electronic module is embedded between said coating on said model and said coating on said electronic module; and
G. providing one or more openings to allow:
a. egress of drugs and fluids in the drug delivery devices;
b. ingress of oral fluids, such as saliva, blood for bio-sensors and monitoring devices;
c. protrusion of the stimulating electrodes to electro-stimulate the muscles and nerves in the electro-stimulator applications suck as the salivary glands stimulator, treatment for snoring and apnea, eating disorder, obesity, dysphagia;
d. optical communication being performed with the intra-oral device via a transparent coating in the visible, Infra Red (IR) or ultraviolet spectrum; and
e. RF communication to pass to/from the device coating allowing wireless communication with the device.

47. A manufacturing method according to Claim 37 that produces a tooth like non-customized devices wherein:
a. creating a module with said electronic module embedded under a coating of material which is a bio-compatible grade material;
b. protecting and sealing said electronic module; and
c. providing one or more openings to allow:
a. egress of drugs and fluids in the drug delivery devices;
b. ingress of oral fluids, such as saliva, blood for bio-sensors and monitoring devices;
c. protrusion of the stimulating electrodes to electro-stimulate the muscles and nerves in the electro-stimulator applications such as the salivary glands stimulator, treatment for snoring and apnea, eating disorder, obesity, dysphagia;
d. optical communication being performed with the intra-oral device via a transparent coating in the visible, Infra Red (IR) or ultraviolet spectrum; and
e. RF communication to pass to/from the device coating allowing wireless, communication with the device.

48. A manufacturing method according to claim 37 that produces a non-customized device resembling a mouth guard, or a denture or a soft tissue retractor further comprising the steps of;
a. creating molds of at least one size with said electronic module embedded under a layer of vinyl, acrylate polymer, silicone or other dental or bio-compatible grade material;
b. protecting and sealing the electronic module;
c. adjusting the device to fit the mouth of a user; and
d. providing one or more openings to allow:
a. egress of drugs and fluids in the drug delivery devices;
b. ingress of oral fluids, such as saliva, blood for bio-sensors and monitoring devices;
c. protrusion of the stimulating electrodes to electro-stimulate the muscles and nerves in the electro-stimulator applications such as the salivary glands stimulator, treatment for snoring and apnea, eating disorder, obesity, dysphagia;
d. optical communication being performed with the intra-oral device via a transparent coating in the visible, Infra Red (IR) or ultraviolet spectrum; and
e. RF communication to pass to/from the device costing allowing wireless communication with tire device.

49. An apparatus for stimulating the salivary glands comprising:
i. a mouthpiece, structured and arranged to detachably engage teeth, and
ii. an appliance comprising:
a. a socket designed to cover at least one tooth;
b. an electrical stimulator circuit associated with the socket, wherein the electrical stimulator produces electrical pulses when activated;
c. a power source unit; and
d. a receiver including a receiver module and a decoding circuit for a remote control.

50. An apparatus according to claim 49 further comprising one or more of the additional following features:
i. a wetness sensor unit designed to sense the intra-oral wetness level, wherein the wetness level received front the wetness sensor selects the desired electro-stimulation level out of pre-defined stimulation patterns;
ii. the commands received item sold receiver select a desired electro-stimulation level out of pre-defined stimulation patterns;
iii. a transmitter unit on the mouthpiece includes a Light Emitting Diode (LED), PP transmitter or any combination thereof;
iv, the receiver unit includes an Infra Red photodetector and receiver modules or a wireless Radio Frequency (RF) based transceiver or a direct contact control or any combination thereof; and
v. sale apparatus does not comprise a transceiver or, alternatively, is coupled to a transceiver.

51. An apparatus according to claim 49 that includes features that enable programming the following parameters: selecting the stimulating electrodes active pair, communication type and speed, patient's specific stimulation pattern, stimulation strength, stimulation voltage, stimulation current, to match his/her personal profile including health history, health status, DNA profile, gender, age, weight or any combination, and wherein the parameters are stored in a nonvolatile memory or battery backup memory.

52. A method for testing an intra-oral device, to be used in humans as well as animals, which has an electronic module, wherein the electronic module is embedded in the device which is made of a bio-compatible material; having at least one opening, stud method comprising the steps of:
i. connecting the Device Under Test (DUT) to a tester;
ii. applying a combination of inputs signals and parameters to the DUT in accordance to a pre-defined scripts;
iii. measuring the functionality of the tested intra oral device and comparing it to pre-defined expected results;
iv. applying pre-defined criterions for ‘pass’ or ‘fail’; and
v. reporting test results.

53. A method according to claim 52 wherein the intra oral device is an intra-oral salivary gland electro-stimulating device; an intra-oral controlled drug delivery device; an intra-oral device for the measurements of blood, saliva and other oral fluids, other analytes of interest, or any combination thereof or an intra-oral device to treat apnea, snoring, sleeping disorders, eating disorder, oropharyngeal dysphagia neurological disorders.

54. A method according to claim 52 wherein the DUT is connected to laboratory equipment.

55. A method according to claim 52 which is performed after manufacturing, before clinical use, at the operation theater, at the clinician clinic or at any combination thereof.

56. A method, according to claim 52 wherein the test procedure may include one or more of the following features:
1. Measuring DUT built-in battery voltage;
2. Measuring DUT built-in battery max drain current:
3. Measuring DUT inputs impedance:
4. Measuring DUT output impedance;
5. Measuring DUT output max current source/sink;
6. Measuring wireless communication sensitivity;
7. Measuring sensors functionality and accuracy;
8. Applying a pre-planned scenarios (52, 61) shown in FIG. 4, including stimulating the DUT inputs and measuring DUT output and comparing them against expected results.
9. Applying several scenarios, simulating real situations, extreme input conditions, varying environmental situations such as high and low temperature, humid and wet;
10. Measuring membrane permeability;
11. Measuring drug delivery mechanism; and
12. Measuring sensors functionality and accuracy.

57. A tester being a salivary gland electro-stimulator tester comprising:
i. an interface to the Device Under Test (DUT);
ii. a state machine;
iii. a testing script; and
iv. input and output devices.

58. A tester according to claim 57 in which the state machine is a microprocessor, an Application Specific IC (ASIC), an electronic module based on off the shelf discrete electronics components or a personal computer.

59. A tester according to claim 57 being connected to a PC (Personal computer) or to a PDA (Personal Digital Assistant) based on a communication protocol.

60. A tester according to Claim 57 banner comprising one or mere of the following features:
A. an intermediate, detachable receptacle for the DUT placed at sockets (37) which connects the DUT and the Test Apparatus (FIG. 2b), allowing sterilization, cleaning, wiping and any combination thereof of the receptacle unit;
B. an additional testing script being based on pre-defined input sequences and comparing the output to the expected results;
C. a test which measures, one or more of the parameters such as: the DUT built-in battery voltage, measuring DUT built-in battery max drain current, measuring DUT inputs impedance, measuring DUT output impedance, measuring DUT output max current source/sink, measuring wireless communication sensitivity, varying environmental situations such as high and low temperature, humid and wet or any combination thereof;
D. upgrading of DUT software or firmware or database being made through, the connection (wired or wireless) to the tester unit;
E. programming the parameters, such as selecting the stimulating electrodes active pair, communication type and speed, patient's specific stimulation pattern, stimulation strength, stimulation voltage, stimulation current, to math his/her personal profile including health history, health status, DNA profile, gender, age, weight or any combination;
F. These configurable parameters are preferably stored in a nonvolatile memory or battery backup memory; and finally
G. the tester reports results, indicating ‘Pass’ or ‘Fail’. A log file specifying the performed test, and Pass/fail indication per test, recommended action and failure description, or any combination thereof, may be produced as an electronic report or print out on paper.

61. A tester according to claim 57 which comprises additional testers in addition to the salivary gland electro-stimulator as follows;
A. a tester for an apparatus of intra-oral, controlled drug delivery device;
B. a tester for an apparatus of intra-oral sensor of biological parameters such as glucose level, blood pressure, heart rate, blood oxidation, nitric oxide, lactate, hemoglobin, blood cells and platelets, triglycerides, cholesterol, INR, BNP, lactate, temperature, pH, pulse, pCO2, pO2, metals, such, as copper, cadmium, markers of cardiac injury, such as troponins T and I, ischemia-modified albumin, fatty acid-binding protein, drugs, such as lithium, naltrexone, or any combination thereof; and
C. a tester for an intra-oral electromuscular stimulation device to treat breathing d disorders, snoring, apnea, eating disorders, oropharyngeal dysphagia neurological disorders or any combination thereof.

62. A tester according to claim 61 wherein tester A further comprises one of the following features:
1. a detachable receptacle for the DUT output which connects the DUT and the Test Apparatus;
2. measuring DUT drug output, measuring DUT drug output minimum level, measuring DUT drug output maximum level, measuring DUT drug level sensor accuracy and resolution, measuring DUT drug flow rate sensor accuracy and resolution;
3. re-programming the following additional parameters: drug delivery pattern, drug delivery schedule, patient's specific drug delivery pattern; and
4. sensors measure the pattern of drug releases during the test session.

63. A tester according to claim 61 wherein tester B comprises one of the following features:
1. measuring sensed parameter sensitivity, sensed parameter accuracy, sensed parameter resolution, sensed parameter tolerance to external interferences;
2. re-programming the following additional parameters, e.g. selecting the measured biological analyte, patient's specific stimulation delivery pattern; and
3. sensors measuring the amount of accuracy of the DUT murmur during the test session and measure the amount measurement resolution of the DUT sensing dining the test session.

64. A tester according to claim 61 wherein tester C further comprises one of the following features:
1. measuring stimulation sequence and delays between the different probes, measuring stimulation sequence and delays between the different probes; and
2. re-programming the following additional parameters selecting the active stimulating electrodes, patient's specific stimulation pattern, stimulation strength, stimulation voltage, stimulation current.
